(19) United States
(12) Patent Application Publication
(10) Pub. No.: US 2008/0213372 A1
(43) Pub. Date: Sep. 4, 2008

(54) Gel and Apparatus for Cleaning and Deodorizing Fluids

(75) Inventors: Jean-Pierre Pastor, Caracas (VE); Evelyne Robert, Bescat (FR)

Correspondence Address: BAKER & DANIELS LLP, 300 NORTH MERIDIAN STREET, SUITE 2700, INDIANAPOLIS, IN, 46204, US

(73) Assignee: BUXAIR N.V., CURACAO (NL)

(21) Appl. No.: 11/632,882

(22) PCT Filed: Jul. 19, 2004
(86) PCT No.: PCT/IB2004/002417

§ 371 Date: Nov. 20, 2007

(51) Int. Cl. A01N 25/08; B01J 19/08; A61L 9/012

(52) U.S. Cl. 424/489; 422/186.04; 424/76.2

(57) ABSTRACT

A gel for cleaning and deodorizing air includes an organic binder with ultraviolet light permeable polymeric molecules and particles of inorganic semi-conductors. The gel includes acrylic molecules, a polar diluent and an ultraviolet light inert charge including particles of silica, of rutile cristalline form of titanium oxide and/or of clay.

The present invention relates to a gel, and an apparatus incorporating said gel, for cleaning and deodorizing fluids, in particular air.

There are numerous air purification systems that have been described, or that are commercially available, aiming to remove various pollutants, such as dust, microparticles, noxious gases, allergens, and pathogenic micro-organisms from the air. Dust and other micro-particles are removed from the air by use of filters, and it is known to remove certain volatile and reactive molecules by the use of catalytic processes. Micro-organisms may be destroyed by ultraviolet light radiation. It is known to combine the use of ultraviolet light radiation and photocatalytic processes to accelerate the degradation of noxious particles and the destruction of microorganisms.

Other existing techniques for the treatment of ambient air include ionization of the air, or by producing ozone. Effective ionization of the air is hard to control and is generally unreliable. Ozone is a very oxidizing species able to kill many microorganisms and degrade volatile compounds, however, it is also unhealthy for the human organism and its use should therefore be avoided in occupied environments.

The use of photo-catalysts in devices for deodorizing or purifying air is described in a number of patent publications, for example, U.S. Pat. No. 5,670,126, U.S. Pat. No. 6,558,639, U.S. Pat. No. 2,002,094,298, U.S. Pat. No. 6,358,374, U.S. Pat. No. 2,004,007,453, JP 11226357, WO 02/085989, and FR 2821558. The most common photo-catalyst used in known systems is titanium dioxide because of its excellent photocatalytic activity when irradiated with ultraviolet radiation and its harmlessness to the human organism. The photocatalyst is generally present within a binder that forms a coating that adheres to a filter element or other surfaces of the air purifying system irradiated by an ultraviolet light source, typically an ultraviolet lamp.

In the prior systems, the titanium dioxide is usually incorporated in an inorganic polymer prepared from monomers of titanium with organic molecules, which are polymerized by mixing the monomers with an organic solvent and an acid or base to form a polymer gel that can be deposited on the article to be coated by dip-coating, spin-coating, or spraying. The coated article is then usually heated to a temperature over 100° C. to complete the reticulation of the polymer coating. The known processes for coating a photocatalytic material on surfaces of various elements are quite time consuming and therefore costly, particularly in relation to articles produced on an industrial scale. Moreover, the heating of the gel in many conventional processes does not allow the implementation of such processes on materials that do not support high temperatures, such as many plastic materials.

The effectiveness of photocatalytic coatings in conventional air purifying systems decreases over time in view of the highly reactive conditions created by the photocatalytic semi-conductor material and its effect on the binder or surface of the support of the photocatalytic coating. The conventional solution to this problem is to use materials in contact with the photocatalytic particles or in the vicinity thereof that are inert and stable, however this increases the cost of such systems particularly due to the complex and time consuming manufacturing steps and the use of expensive materials.

In view of the aforegoing, it is an object of this invention to provide a versatile, economic, reliable and effective gel for photocatalytic treatment of fluids, in particular air.

It is a further aim to provide a photocatalytic gel that is easy to prepare and handle, in particular that can be easily stored, transported, and applied to articles to be coated.

It is also an object of this invention to provide an apparatus incorporating a photocatalytic gel for purification of air, that is economic to manufacture, versatile, reliable, and effective.

It is a further aim to provide a apparatus that is easy and economical to use and maintain.

Objects of this invention have been achieved by providing a photocatalytic gel according to claim 1.

Disclosed herein is a gel for cleaning and deodorizing air, that includes an organic binder including ultraviolet light permeable polymeric molecules and particles of inorganic semi-conductors. The polymeric molecules advantageously include acrylic molecules. Acrylic molecules are particularly convenient and efficient for manufacturing the gel according to the present invention.

Advantageously, the incorporation of an inorganic non-toxic semi-conductor into an organic binder comprising a polymeric matrix permeable to ultraviolet light rays provides a gel that is easy to use, handle and store, exhibits good adhesion to many types of materials, and can be applied on articles that do not support high temperatures.

An important advantage of the acrylic-based binder is that it may be mixed with the semi-conductor powder and the diluent and any other additives at room temperature and coated on the surface of a support with known coating techniques. The acrylic-based gel has good adhesive properties to a large number of surfaces, including polymer surfaces and moreover can be dried in air without high temperatures, thus allowing the coating to be used on materials that do not support high temperatures, such as various plastic materials. The acrylic-based gel may also be easily stored and handled in hermetic opaque containers for long periods of time in view of the stability of the mixture.

A further advantage of the acrylic-based gel is that it is permeable to ultraviolet light and thus allows the ultraviolet light to efficiently act on the photo-sensitive semi-conductor particles set in the binder.

According to a particular feature of the invention, the inorganic semi-conductor particles preferably have an average diameter smaller than 100 nm. This provides a high global specific surface of photo-catalyst material.

The gel may include a polar diluent, preferably water, which helps to reduce the density of the polymeric particles. The ratio of the weight of the diluent to the weight of the organic binder is less than 20. The ratio of the weight of the diluent to the weight of the organic binder is preferably less than 10. This feature improves the ease of manipulation of the gel and ensures a good consistency for applying a thin coating of gel on the catalyser.

The gel may further include an ultraviolet light inert charge. The inert charge partially reflects light rays and thus reduces the risk of damaging the polymeric molecules. The weight of the inert charge is preferably less than 30% of the weight of both the diluent and the binder, which allows both the protection of the polymeric molecules and the transmission of ultraviolet light rays to the particles of semi-conductor. The inert charge may include particles of silica, of rutile cristalline form of titanium oxide and/or of clay.

Preferably, the weight of the semi-conductor particles is 2 to 10 times less than the weight of both the diluent and the binder. This proportion gives good results in terms of gel density and thus the ease of preparing and manipulating the gel, whilst also offering efficient photocatalysis.

The photocatalytic semi-conductor particles may include cadmium sufilde, zinc sulfide and/or titanium dioxide in anatase cristalline form, which all exhibit high photocatalysis effects. Preferably, the photocatalytic semi-conductor used in the present invention is titanium dioxide because of its effective catalytic activity in the presence of ultraviolet light, in addition to its stability and harmlessness to the human organism.

The gel may further be impregnated by metal, for example, iron, magnesium, and/or vanadium. These metals enlarge the absorption band of the gel towards the visible domain, thus improving photocatalysis.

Objects of this invention have also been achieved by providing an apparatus for cleaning and deodorizing air according to claim 10.

Disclosed herein is an apparatus for cleaning and deodorizing air, comprising a catalyser tube, an ultraviolet light source received in the catalyser tube for irradiating an inner surface thereof, a housing, and an airflow system for propelling or drawing air through the catalyser tube, wherein the catalyser tube is removably mounted in the apparatus housing and is coated on the portion of its inner surface that is irradiated with ultraviolet light with a photocatalytic gel comprising an organic binder with ultraviolet light permeable polymeric molecules and particles of an inorganic photosensitive semi-conductor. The apparatus may further comprise a filter system at its inlet for dust and large objects

The gel may advantageously comprise any or all of the further characteristics described hereinabove.

The catalyser tube may advantageously be made of a low cost polymer-based material that could be made in a tube shape for example by injection moulding, or extrusion, or from a sheet that is folded into a tube with the desired profile and fixed along the seam thereof. The sheet may be made from a laminated polymer or metal material, or from a woven fibre such as woven fibreglass. Woven fibreglass is particularly advantageous since it is resistant to UV light and the catalytic action of the photocatalytic semiconductor, and is very economic to manufacture. The fibreglass also presents a very good support for adhesion of the coating. The catalyser tube according to this invention may thus be easily and economically replaced at regular intervals, for example annually. The ability to easily and economically change the catalyser tube enables the apparatus to have a long operation lifetime, while at the same time ensuring effective deodorizing and cleaning properties in a very economic manner. The ability to use common lightweight materials for the catalyser tube allows the apparatus to be light and versatile, and to be implemented in portable or fixed apparatuses for domestic or industrial use.

Advantageously, the catalyser tube can be provided with a relatively large surface in comparison to the ultraviolet light source and with a reasonable length to ensure effective contact with the air flowing therethrough. The distance between the inner irradiated surface of the catalyser tube is preferably between 1 cm to 3 cm from the ultraviolet light source in order to optimize the overall effectiveness of the cleaning and deodorizing activity of the apparatus by optimizing the balance between the intensity of the photocatalytic activity of the gel and the direct germicidal action of the UV light with the surface area of the photocatalytic gel in contact with the air. The catalyser tube may be provided with a generally prismatic cylindrical shape, or with other profiles, depending on the arrangement of the ultraviolet light source and the projection of light rays therefrom.

A more complete appreciation of the present invention and many of its advantages, aims and characteristics will be further understood from the claims and the following detailed description, in connection with the accompanying drawings, in which

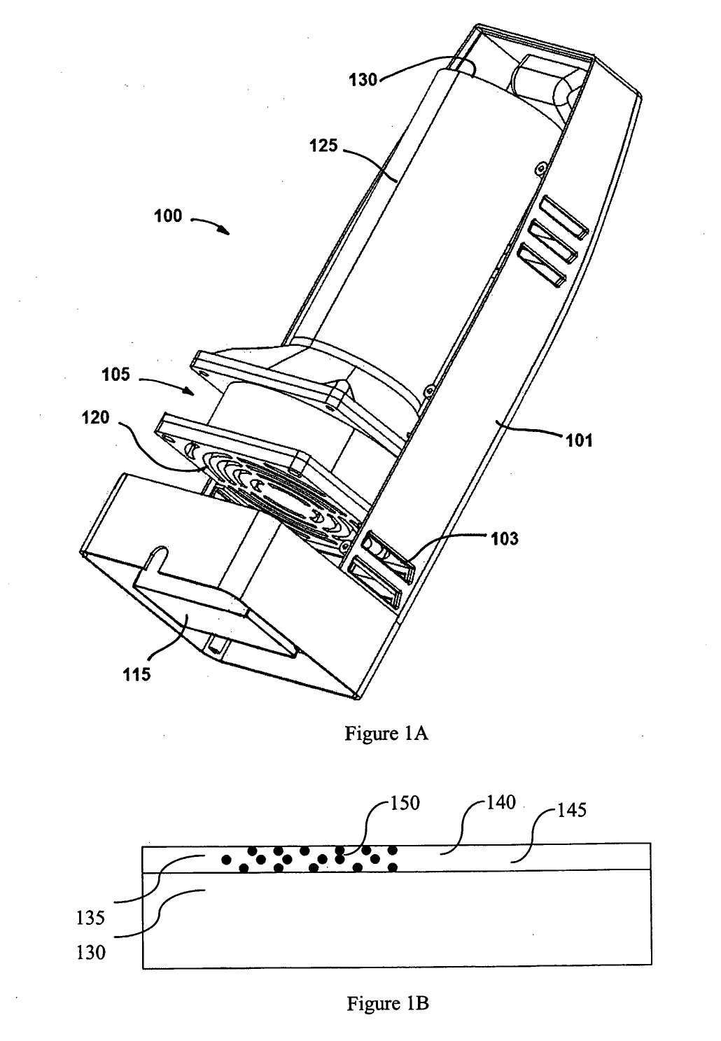

FIG. 1A is a perspective view of an apparatus for cleaning and deodorizing air according to the present invention, with a portion of the housing removed;

FIG. 1B is an enlarged view of part of a support and a gel of the apparatus lustrated in FIG. 1;

FIG. 1C is an exploded perspective view of the apparatus of FIG. 1 without the housing; and

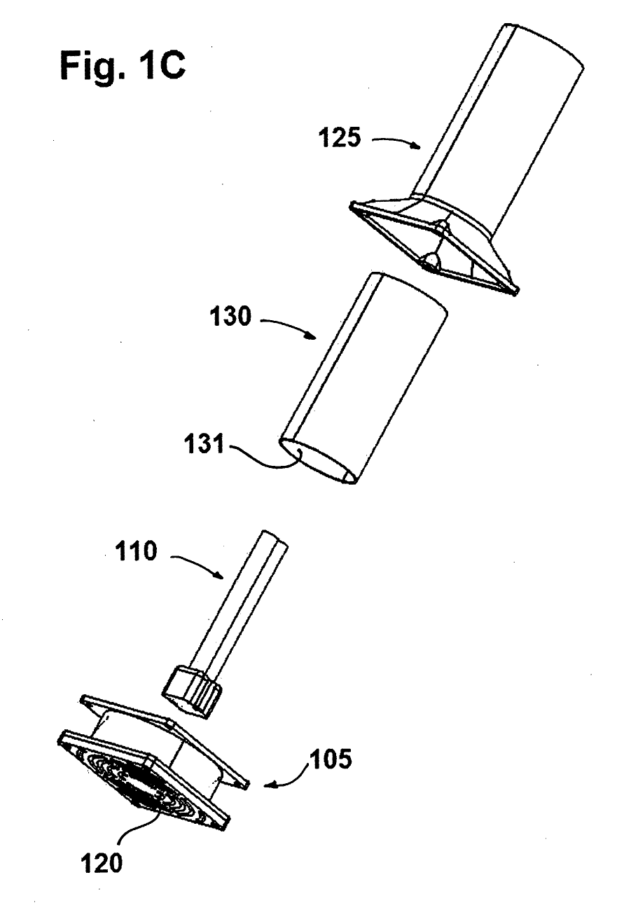

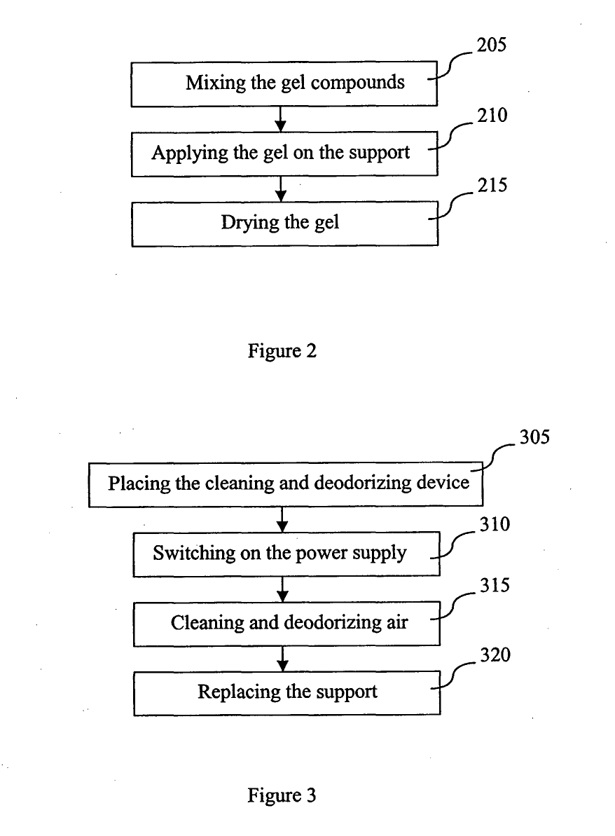

FIGS. 2 and 3 are schematic flowcharts of the steps for manufacturing and using the apparatus shown in FIG. 1.

Ultraviolets rays (“UV”) are able to destroy or repel bacterias, viruses and other micro-organisms. The absorption of UV rays energy modify groups of nucleic acid molecules and thus disturbs the information carried by those molecules and the duplication of micro-organisms DNA. The reproduction of micro-organisms or their division being impossible, the death of the micro-organisms is inevitable. These processes have been widely studied.

Irradiation of a photocatalytic semi-conductor in the presence of oxygen generates very active oxidative species capable of degrading organic pollutants and odor compounds until their mineralization, i.e. their transformation into carbon dioxide and water.

When a semi-conductor particle absorbs light having at least the band gap energy of the semi-conductor material, electrons present in the valency electron band are excited to migrate to the conduction band thus creating oxidation sites and reduction sites. These sites react with water and oxygen to generate hydroxyl and superoxide radicals, that are decomposing agents for organic compounds. The present invention implements a photosensitive semi-conductor in a polymeric material that resists ultraviolet light to clean and deodorize the ambient air.

FIGS. 1A to 1C illustrate an embodiment of the apparatus 100 for cleaning and deodorizing air according to the present invention. The apparatus 100 includes a housing 101, an airflow system 105, a power-supply 115, a filter 120, a removable catalyser tube 130 mounted in a conduit 125, and an ultraviolet light source 110 positioned within the catalyser tube. In this embodiment, the apparatus forms a portable or moveable unit that can be removably installed in a room in a domestic or industrial environment.

The catalyser tube may advantageously be made of a polymeric material, for example as a rigid moulded or extruded plastic element, or flexible sheet of laminated or woven fibre material folded and bonded along a seam to form a tube. The polymer may be polyethylene, polybutadieneterephtalate, polybutadienestyreneacrylonitrile, polymethyl methacrylate, polyvinylchloride or fibre-glass mineral or cellulosic types of polymers. The catalyser tube may also be made of other cost effective materials such as sheet metal bent into a tube and welded, crimped or bonded along its seam. For embodiments where the catalyser tube is made from a sheet of flexible material, the sheet may even be supplied from a roll of material, cut into sections of appropriate length, or as an essentially flat sheet, that is bent into a tube at the time of its insertion into the conduit, and held in place against the wall of the conduit either with its own elasticity or with fixing clips or other fixing means provided in the conduit. The flexible catalyser sheet can thus be coated with gel before it is bent into a tube to facilitate manufacturing thereof. The flexible catalyser sheets may also be coated in a substantially continuous and thus low cost process if desired.

At least the interior surface of the catalyser tube, which is irradiated with UV light from the UV light source, is coated with a thin layer of photocatalytic material 135.

The ultraviolet light source 110, which is preferably positioned substantially along the central axis of the catalyser tube, generates light having a wavelength between 180 and 400 nm, preferably between 240 and 420 nm. The light source 110 may be one or more UV lamps. It can a low or medium pressure mercury lamp, an incandescent lamp or a fluorescent lamp. It may have a cylindrical shape, a bulb shape or any other shape.

The airflow system 105, which may comprise a blade fan driven by an electric motor, draws air through the housing inlet duct 103 and the filter system 120, and blows the air through the removable catalyser tube 130, in which the ultraviolet light source 110 is positioned. The filter 120 removes insects, large particles and dust. The removable catalyser tube 130 is positioned in a conduit 125 that is a fixed component to which the ultraviolet light source 110 is mounted and to which the airflow system and filter are mounted. The removable catalyser tube 130 is positioned in the conduit so that its surface coated with the photocatalytic layer receives as much irradiating light rays as possible from the ultraviolet light source 110. The removable catalyser tube 130 may be easily accessed by taking off a housing part 104, and subsequently removed and replaced by another identical removable catalyser tube. The removable catalyser tube may have a simple prismatic shape, or may take different forms according to different shapes or different uses of the apparatus according to the present invention.

In the embodiment shown in FIGS. 1A to 1C, the layer of photocatalytic material or photocatalyser 135 is bonded on the inner surface of the removable catalyser tube 130 by an ultraviolet light permeable organic binder 140.

The layer of photocatalytic material is in the form of a gel comprising the organic binder 140 including ultraviolet light permeable polymeric molecules 145 and particles of inorganic semi-conductor 150. Preferably, the polymeric molecules 145 are acrylic molecules. An important feature of the organic binder according to this invention is that it is easy to use and exhibits good adhesion to many types of materials.

The polymeric gel containing the particles of semi-conductor is applied onto an inner surface 131 of the removable catalyser tube 130 in order to use its photocatalytic properties to clean and deodorizing air. Preferably, the thickness of the gel layer is less than 1 mm on the surface of the removable catalyser tube 130.

The specific area of the particles of semi-conductor is between 40 and 200 m²/g.

Preferably, the gel is impregnated by metal, such as iron, magnesium and/or vanadium, that enlarges the absorption band towards the visible domain.

The polymeric gel may further include a polar diluent, for example water, the ratio of the weight of the polar diluent to the weight of organic binder being between less than 20 and preferably less than 10.

The photocatalyser is a semi-conductor of at least one of the following types, cadmium sulfide, zinc sulfide or titanium dioxide in anatase cristalline form. Preferably, the particules of inorganic semi-conductor 150 exhibit an average diameter smaller than 100 nm. The weight of the inorganic semi-conductor particles 150 is 2 to 20 times less than the weight of both the binder and the diluent, preferably 3 to 15 times less.

Preferably, the polymeric gel incorporates a UV light inert charge that includes particles of silica, of titanium oxide in rutile cristalline form or of clay, those particles representing less than 30% of the weight of both the binder and the diluent.

The apparatus 100 uses two physico-chemical processes for cleaning and deodorizing air and two complementary technologies: the germicidal action of ultraviolet light rays and the mineralization via photocatalysis. Altogether, those two technologies actively fight against pathogenic and/or allergenic micro-organisms as well as against volatile organic compounds originating from the industrial pollution apparatus 100 allows, by combining the afore-mentioned technologies, to efficiently purify ambient air by destroying micro-organisms, volatile organic compounds as well as odor compounds. The apparatus may be used as an home appliance or for professional purpose as well, according to its size.

An example of preparation of the gel is as follows:

40 parts (in weight) of water are added to 60 parts of an acrylic binder under agitation. Then, a mixture of 15 parts of silica and 10 parts of titanium dioxide are added to the gel under continuous stirring until a homogeneous gel is obtained. The liquid gel may be stored for a long time (many months) without degradation, at ambient temperature and kept in a container that is hermetic to light and air.

FIG. 2 is a schematic flowchart of the steps for manufacturing the apparatus 100. During step 205, the gel compounds, as listed above with regard to FIGS. 1A to 1C, are mixed. The resulting solution is a white gel. During step 210, the gel is applied onto the inner surface of the removable catalyser tube 130. That may be done by pulverization, dip coating, spin coating or brush coating. During step 215, drying of the gel may advantageously be performed at ambient temperature, by hot air ventilation or by infrared heating at relatively low temperatures (less than 100° C.), in order to obtain a thin layer of gel having a thickness less than 1 mm.

FIG. 3 is a schematic algorithm of the steps for using the apparatus 100 illustrated in FIGS. 1A to 1C as manufactured according to the steps shown in FIG. 2. During step 305, the apparatus is placed in a professional or home room. During step 310, the power supply 115 is connected to the room main electrical power supply and is switched on. When the power supply 115 is switched on, the fan 105 blows ambient air into the support 125 and the removable catalyser tube 130 receiving UV light rays from the ultraviolet light source 110. As long as the power supply 115 is switched on, step 315, the air blown by the fan 105 passes through the filter 120 and then into the removable catalyser tube 130, where it is irradiated by ultraviolet light rays, and on the photocatalyser 135. The air is thus cleaned and deodorized as explained above. By leaving the apparatus on in a confined volume, the blown ambient air recirculates through the catalyser tube and ensures the degradation of any volatile compounds or pathogens remaining after a first pass through the catalyser tube.

After a predetermined time period of use, depending on the use of the apparatus, e.g., every twelve months, during step 320, the removable catalyser tube 130 is removed and replaced by another identical removable catalyser tube.

Thus, the present invention is easy to implement and easy to use, even by non-professionals.

1. Gel for cleaning and deodorizing air, comprising an organic binder comprised of ultraviolet light permeable polymeric molecules, a polar diluent, and particles of inorganic semi-conductor, the weight of the semi-conductor particles being two to twenty times less than the weight of both the diluent and the binder.

2. Gel according to claim 1, wherein the inorganic semi-conductor particles have an average diameter smaller than 100 nm.

3. Gel according to claim 1, wherein the polymeric molecules include acrylic molecules.

4. Gel according to claim 1, wherein the ratio of the weight of the diluent divided by the weight of the organic binder is less than 20.

5. Gel according to claim 1, wherein the gel includes an ultraviolet light inert charge.

6. Gel according to claim 5, wherein the weight of the inert charge is less than 30% of the weight of both the diluent and the binder.

7. Gel according to claim 5, wherein the inert charge includes particles of silica, of rutile crystalline form of titanium oxide and/or of clay.

8. Gel according to claim 1, wherein the semi-conductor particles include cadmium sufilde, zinc sulfide and/or titanium dioxide in anatase crystalline form.

9. Gel according to claim 1, wherein the gel is impregnated by a metal to enlarge the absorption band towards the visible domain.

10. Apparatus for cleaning and deodorizing air, comprising a catalyser tube, an ultraviolet light source received in the catalyser tube for irradiating an inner surface thereof, a housing, and an airflow system for propelling or drawing air through the catalyser tube, wherein the catalyser tube is removably mounted in the apparatus housing and is coated on a portion of its inner surface that is irradiated with ultraviolet light with a photocatalytic gel comprising a binder and particles of an inorganic photosensitive semi-conductor.

11. Apparatus according to claim 10, wherein the photocatalytic gel comprises an organic binder including ultraviolet light permeable polymeric molecules, a polar diluent, and particles of inorganic semi-conductor, the weight of the semi-conductor particles being two to twenty times less than the weight of both the diluent and the binder.

12. Apparatus according to claim 11, wherein the inorganic semi-conductor particles have an average diameter smaller than 100 nm.

13. Apparatus Gel according to claim 11, wherein the polymeric molecules include acrylic molecules.

14. Apparatus according to claim 11, wherein the ratio of the weight of the diluent divided by the weight of the organic binder is less than 20.

15. Apparatus according to claim 11, wherein the gel includes an ultraviolet light inert charge.

16. Apparatus according to claim 15, wherein the weight of the inert charge is less than 30% of the weight of both the diluent and the binder.

17. Apparatus according to claim 15, wherein the inert charge includes particles of silica, of rutile crystalline form of titanium oxide and/or of clay.

18. Apparatus according to claim 11, wherein the semi-conductor particles include cadmium sufilde, zinc sulfide and/or titanium dioxide in anatase crystalline form.

19. Apparatus according to claim 11, wherein the gel is impregnated by a metal to enlarge the absorption band towards the visible domain.

20. Apparatus according to the claim 10, wherein the catalyser tube is made of a woven fiber material.

21. Apparatus according to the claim 20, wherein the fiber material is fiber glass.

22. Apparatus according to claim 10, wherein the catalyser tube has a generally prismatic shape.

23. Apparatus according to claim 10, wherein the ultraviolet light source is in the form of one or more UV light tubes removably mounted substantially along the central axis of the catalyser tube.

24. Apparatus according to claim 10, wherein the inner surface of the catalyser tube is positioned at a distance between one to three centimetres from the ultraviolet light source.

25. Apparatus according to claim 10, further comprising a filter system at an inlet of the housing for filtering dust and large particles.

26. Apparatus according to claim 25, wherein the airflow system comprises a blade fan driven with an electric motor.

27. Apparatus according to any claim 26, in the form of a portable or movable unit.
